(19) United States
(12) Patent Application Publication
(10) Pub. No.: US 2023/0080043 A1
(43) Pub. Date: Mar. 16, 2023

(54) AUTOMATED CARDIAC DEFIBRILLATOR PACER WITH INTEGRATED CARDIAC ASSIST DEVICE

(71) Applicant: NewPace Ltd., Caesarea (IL)

(72) Inventors: Robert Fishel, Caesarea (IL); Gera Strommer, Caesarea (IL); Avi Broder, Caesarea (IL)

(21) Appl. No.: 17/793,478

(22) PCT Filed: Jan. 20, 2021
(86) PCT No.: PCT/IB2021/050410

§ 371 Date: Jul. 18, 2022

Related U.S. Application Data
(60) Provisional application No. 62/976,473, filed on Feb. 14, 2020.
(60) Provisional application No. 62/963,169, filed on Jan. 20, 2020.

(51) Int. Cl. A61N 1/39

(52) U.S. Cl. CPC A61N 1/3904; A61N 1/39622

(57) ABSTRACT

Percutaneous transvenous defibrillator and/or pacing devices with integrated cardiac assist devices and method of use. In some embodiments, a device may comprise a shared catheter, a defibrillator assembly, an automated external defibrillator (AED) and a cardiac assist assembly, wherein the defibrillator assembly includes at least two defibrillation coils in communication with the AED and wherein the defibrillator assembly and the cardiac assist assembly use the shared catheter for percutaneous and intravenous implantation into a patient. In some embodiments, a device may comprise a shared catheter, a pacing assembly, a pacing controller and a cardiac assist assembly, wherein the pacing assembly includes at least two electrodes in communication with the pacing controller, and wherein the pacer assembly and the cardiac assist assembly use the shared catheter for percutaneous and intravenous implantation into a patient.

### CROSS REFRENCE TO RELATED APPLICATIONS

This is a 371 application from international patent application PCT/IB2021/050410 filed Jan. 20, 2021, and is related to and claims priority from U.S. provisional patent applications No. 62/963,169 filed 20 Jan. 2020 and 62/976,473 filed 14 Feb. 2020, both of which are incorporated herein by reference in their entirety.

### FIELD

Embodiments disclosed herein relate to automated cardiac defibrillators and integrated cardiac assist devices.

### BACKGROUND

A ventricular assist device or cardiac assist device provides cardiac assist functionality. Non-limiting examples of such cardiac assist devices include cardiac assist pumps, pacemakers, heart monitors, cardiac central pressure monitor, cardiac oximetry sensor and so forth. For example, a cardiac assist pump is an electromechanical device that assists in cardiac circulation to partially or to completely replace the pumping function of a failing heart. Current technology allows for insertion of small axially driven cardiac assist pumps into the heart. Some cardiac assist devices are built in a form of a catheter pump inserted percutaneously, typically via the femoral artery, into the ascending aorta, across the valve and into the left ventricle. Once in position inside the heart, the cardiac assist device draws blood out of the left ventricle and pumps it into the ascending aorta thereby adding pressure to this blood flow. Cardiac assist devices can be used to support both the right ventricle and the left ventricle. Instances of use of these types of devices include high risk angioplasty, acute coronary syndromes such as acute myocardial infarction and weaning of the heart from the heart lung machine after open heart surgery. Non-limiting examples of such cardiac assist devices in a form of a pump catheter are the Abbott HeartMate® PHP, the AbioMed Impella® and the Terumo iVAC2L™.

Cardiac arrhythmias such as ventricular tachycardia (VT) and ventricular fibrillation (VF) are common causes of death, especially in patients with heart failure or in the period immediately surrounding acute coronary occlusions or coronary interventions.

Many patients who undergo cardiac assist device implantation suffer from malignant ventricular arrhythmias such as VT or VF and require external electrical shocks from a defibrillator to restore a normal heart rhythm. These shocks are typically delivered using a standard automated external defibrillator (AED) in which a high energy, high voltage shock is initiated by the AED after personnel operating the defibrillator position the AED patches on the patient's body. Patients in this state may require cardiopulmonary resuscitation (CPR) but the need for resuscitation is often not identified immediately and the delivery of an external defibrillating shock is thus delayed. This delay in recognition and delivery of defibrillation therapy for treating VT/VF in these patients causes both increased morbidity and mortality.

A wearable automatic defibrillator such as the LifeVest® device manufactured by Zoll Medical® can be offered to such patients but currently is not used as patients with cardiac assist devices are not typically ambulatory. In the future however, longer term use of cardiac assist devices will require these patients, all of whom are at increased risk of arrhythmic death, to be protected from arrhythmic sudden death with immediately available defibrillation. Many such patients do not already have an internal cardiac defibrillators (ICD) prior to their cardiac assist device insertion. Furthermore, current Centers for Medicare & Medicaid Services (CMS) guidelines do not allow implantation of a permanent ICD in these patients until after a mandated waiting period, which can be as long as 3 months after a coronary intervention. Thus, there is a real clinical need for patients with cardiac support devices to have back-up rescue automatic defibrillation.

Further, patients who need a cardiac assist device typically suffer from or are at risk of conduction system disease including complete heart block. For example, an acute anterior wall myocardial infarction can result in necrosis of the conduction system and cause cardiac asystole. In addition, many patients with active heart failure often will already have a left bundle branch block (LBBB) and resultant ventricular desynchrony. In addition, insertion of a cardiac assist device in a patient with a pre-existing right bundle branch block (RBBB) can result in trauma to the remaining left bundle branch and cardiac asystole. Finally, insertion of a right sided cardiac assist device in a patient with a LBBB can in turn traumatize the remaining right bundle branch and also result in cardiac asystole.

### SUMMARY

Exemplary embodiments disclosed herein relate to a percutaneous transvenous defibrillator and/or pacing device with an integrated cardiac assist device and method of use. More specifically, different aspects disclosed herein provide for one or more external devices attached to an insertable assembly having internal components, such as electrodes and sensors, that may provide sensing, pacing and/or defibrillation as well as cardiac assist functionality. The internal components for providing sensing, pacing and/or defibrillation are advantageously compact components for integration with internal cardiac assist functionality.

Embodiments disclosed herein may be configured to provide variations of the internal components, external devices and cardiac assist functionality and include: a percutaneous transvenous defibrillator with an integrated cardiac assist device or “defibrillating cardiac assist device” (DCAD), a percutaneous transvenous pacer with an integrated cardiac assist device or “pacing cardiac assist device” (PCAD), or a combined “defibrillating and pacing cardiac assist device” (DPCAD). In some embodiments, an insertable percutaneous transvenous defibrillator/pacer (IDP) may be used with an implanted cardiac assist device. In the disclosed embodiments the cardiac assist device/component may be any of a cardiac assist pump, a pacemaker, a heart monitor, a cardiac central pressure monitor, a cardiac oximetry sensor and so forth.

In some embodiments, the DCAD, DPCAD or IDP includes an external AED and internal defibrillator assembly having high voltage anode and cathode coils and for performing sensing of ventricular arrhythmias and for automated delivery of defibrillation shocks provided by the external AED during periods of detected VT/VF.

In some embodiments, a PCAD, DPCAD or IDP includes an external pacing controller and internal pacing assembly having unipole and bipole electrode configurations. In some embodiments, a PCAD, DPCAD or IDP includes unipolar electrodes that may be positioned at the distal end of the cardiac assist device, at the aortic arch (or IVC/RA/RV in the case of a right sided cardiac assist device), and in the descending aorta. In some embodiments, a PCAD, DPCAD or IDP includes a distal bipolar pacing electrode pair that may be positioned at the very distal portion of the cardiac assist device. In some embodiments, a PCAD, DPCAD or IDP includes a dedicated screw in pacing lead originating at the distal end of the cardiac assist device and screwed into an appropriate location in the left ventricle (LV) or right ventricle (RV).

In some embodiments, an IDP utilizes the catheter and assist port of an implanted cardiac assist device for implanting of the defibrillator assembly and/or pacer assembly into a patient.

Integration of automated defibrillator and/or pacer functionality with cardiac assist functionality may provide several advantages and alternate functionality including:
Single implantation procedure for all devices;
Single device and single exit port from patient's body reduces discomfort such as when wearing a wearable defibrillator;
Fast automated detection of VT/VF and immediate activation of defibrillation shocks;
Rapid detection of ventricular tachycardia via intracardiac sensing;
Discrimination of VT from atrial arrhythmias with aberration via sensing of atrial activity utilizing an atrial sensing dipole place on the shaft of the device;
Lower defibrillating thresholds are possible using the internal shocking coils than possible via external AEDs thus resulting in lower concurrent pain and patient trauma from a given shock;
Ability to sense whether a given arrhythmia results in hemodynamic compromise using pressure sensors integrated the defibrillating cardiac assist device;
Ability to deliver anti-bradycardia pacing of ventricular tachycardia via the distal electrode of the device as an anode and either a common ground or another cathodal electrode on a more proximal section of the device;
Ability to deliver anti-tachycardia pacing of ventricular tachycardia via the distal electrode of the device as an anode and either a common ground or another cathodal electrode on a more proximal section of the device;
Anti-tachycardia pacing delivery via any unipolar or bipolar pacing pair to help terminate monomorphic VT prior to degeneration into ventricular fibrillation;
Dynamically anticipative resynchronization pacing of either the LV or RV (in the setting of a right bundle branch block) using sensing from either surface electrodes or any of the various unipolar and/or bipolar electrodes integrated into the device;
Integrated LV/RV pacing and high voltage shocking therapy using the same high voltage coil electrodes in the heart as a source of power for both pacing and shock delivery;
Selectable shocking and pacing bipoles including pacing and shocks between the distal coil or electrode pair of the cardiac assist device and the coils in the more proximal portions of the device which, in an LV placement, would be at the aortic arch and descending aorta;
P-wave and QRS complex sensing via various electrode unipolar and bipolar pairs to find a combination with optimized P and QRS sensing for effective anticipative resynchronization pacing;
An external automated pacing controller capable of sensing malignant ventricular and atrial arrhythmias and delivering automated high voltage therapy, anti-tachycardia pacing, backup pacing and resynchronization pacing as needed by the individual patient;
An external pacing controller with automated electrode pair selection for ideal pacing efficiency or hemodynamic benefit;
An external pacing controller with automated electrode pair selection for ideal sensing of P wave for atrial activity and QRS complexes for ventricular activity including ventricular capture during pacing;
An implantable device with automated electrode pair selection for ideal pacing efficiency or hemodynamic benefit;
An implantable device with automated electrode pair selection for ideal sensing of P wave for atrial activity and QRS complexes for ventricular activity including ventricular capture during pacing.

In various embodiments, there is provided a device, comprising a shared catheter, a defibrillator assembly, an AED and a cardiac assist assembly, wherein the defibrillator assembly includes at least two defibrillation coils in communication with the AED and wherein the defibrillator assembly and the cardiac assist assembly use the shared catheter for percutaneous and intravenous implantation into a patient.

In some embodiments, the device further comprises an external pump controller, wherein the cardiac assist assembly comprises an internal flow pump adapted for pumping of blood from the patient's heart, and wherein the internal flow pump is in communication with the external pump controller.

In some embodiments, the defibrillator assembly further comprises at least one cardiac rhythm sensor in communication with the AED In some embodiments, the device further comprises at least one pressure sensor in communication with the AED. In some embodiments, the AED is configured to detect sustained ventricular arrhythmia in the heart of the patient and to deliver a high voltage shock via the at least two defibrillation coils when the sustained ventricular arrhythmia is detected. In some embodiments, the arrhythmia detection is based on a signal sensed from the at least two defibrillation coils, from the at least one cardiac rhythm sensor or the at least one pressure sensor. In some embodiments, the at least one cardiac rhythm sensor comprises a first cardiac rhythm sensor and a second cardiac rhythm sensor.

In some embodiments, the cardiac assist assembly is selected from the group consisting of a heart monitor, cardiac central pressure monitor, and a cardiac oximetry sensor.

In various embodiments, there is provided a device, comprising a shared catheter, a pacing assembly, a pacing controller, and a cardiac assist assembly, wherein the pacing assembly includes at least two electrodes in communication with the pacing controller, and wherein the pacer assembly and the cardiac assist assembly use the shared catheter for percutaneous and intravenous implantation into a patient. In some embodiments, one of the at least two electrodes is a pacing lead. In some embodiments, the pacing lead is a screw-in pacing lead. In some embodiments, the device is configured to use the at least two electrodes for both pacing and sensing.

In some embodiments, the device further comprises an external pump controller, wherein the cardiac assist assembly comprises an internal flow pump adapted for pumping of blood from the patient's heart, wherein the internal flow pump is in communication with the external pump controller.

In some embodiments, the pacing controller is configured for selecting an electrode pair for pacing and an electrode pair for sensing.

In some embodiments, the device further comprises an external electrode for adhering to the skin of the patient. In some embodiments, any combination of the at least two electrodes and the external electrode may be used to form a common ground.

In some embodiments, the device is configured to provide dynamically anticipative resynchronization pacing.

In some embodiments, the device further comprises an AED, wherein at least two of the electrodes are high voltage (HV) electrodes, wherein the AED is in communication with the HV electrodes and configured to use the HV electrodes to provide defibrillation. In some embodiments, the AED is configured to detect a sustained ventricular arrhythmia in the heart of the patient and to deliver a high voltage shock via the HV electrodes when the sustained ventricular arrhythmia is detected. In some embodiments, the AED is integrated into the pacing controller or vice versa.

In some embodiments, the pacing assembly further comprises at least one cardiac rhythm sensor in communication with the pacing controller. In some embodiments, the detection of the sustained ventricular arrhythmia is based on a signal sensed from one or more of the HV electrodes, the at least one cardiac rhythm sensor, and the at least one pressure sensor. In some embodiments, the at least one cardiac rhythm sensor comprises a first cardiac rhythm sensor and a second cardiac rhythm sensor.

In some embodiments, the device further comprises at least one pressure sensor in communication with the AED.

In an embodiment, there is provide a device for use with a cardiac assist device for implantation into a patient, comprising a pacing and defibrillation assembly, an AED and a pacing controller, wherein the AED and pacing controller are in data communication with the pacing and defibrillation assembly for providing sensing, pacing and defibrillation, wherein the cardiac device comprises a catheter and an assist port and wherein the pacing and defibrillation assembly is percutaneously and intravenously implanted into the patient using the catheter and the assist port of the cardiac device.

This summary is provided to introduce a selection of concepts in a simplified form that are further described below in the detailed description. This summary is not intended to identify key features or essential features of the claimed subject matter, nor is it intended to be used to limit the scope of the claimed subject matter.

Unless otherwise defined, all technical and scientific terms used herein have the same meaning as commonly understood by one of ordinary skill in the art. The materials, methods, and examples provided herein are illustrative only and not intended to be limiting.

### BRIEF DESCRIPTION OF THE DRAWINGS

Aspects, embodiments and features disclosed herein will become apparent from the following detailed description when considered in conjunction with the accompanying drawings. Like elements may be marked with like numerals in different figures, where:

FIG. 1A shows an exemplary illustrative drawing of a DCAD and FIGS. 1B-1D show exemplary illustrative drawings of a DCAD implanted in a patient according to some embodiments;

FIGS. 2A and 2B show exemplary illustrative drawings of an IDP installed into a cardiac assist device according to some embodiments;

FIG. 3 shows an exemplary illustrative drawing of an IDP integrated into a cardiac device according to some embodiments;

FIGS. 4A-4D show exemplary illustrative drawings of a DPCAD implanted in a patient according to some embodiments;

FIG. 5 shows an exemplary illustrative drawing of an IDP integrated into a cardiac device according to some embodiments.

### DETAILED DESCRIPTION

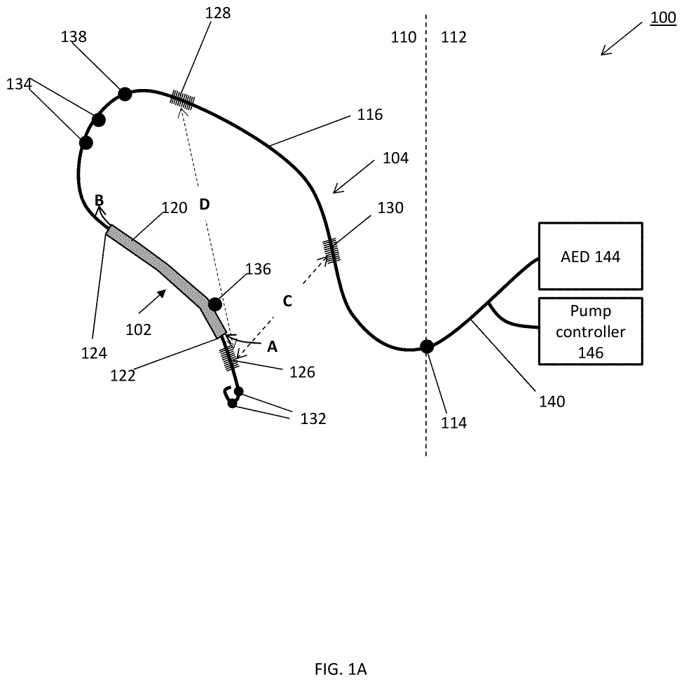

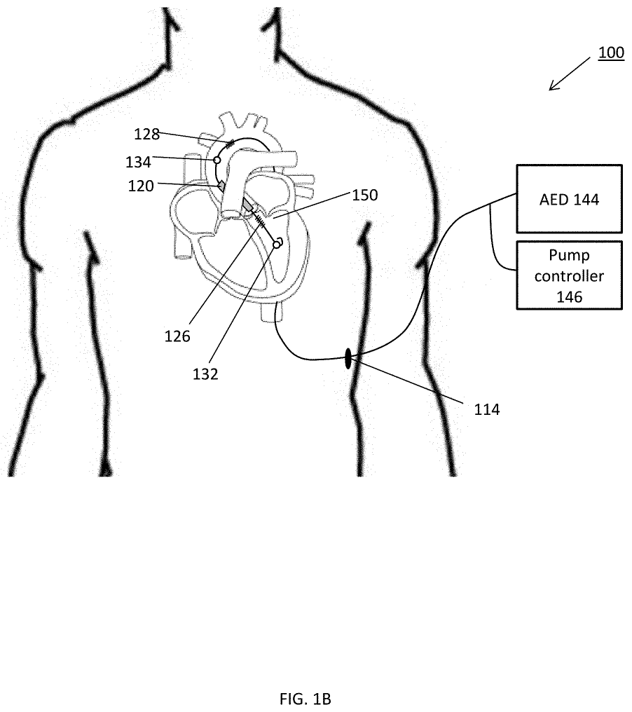

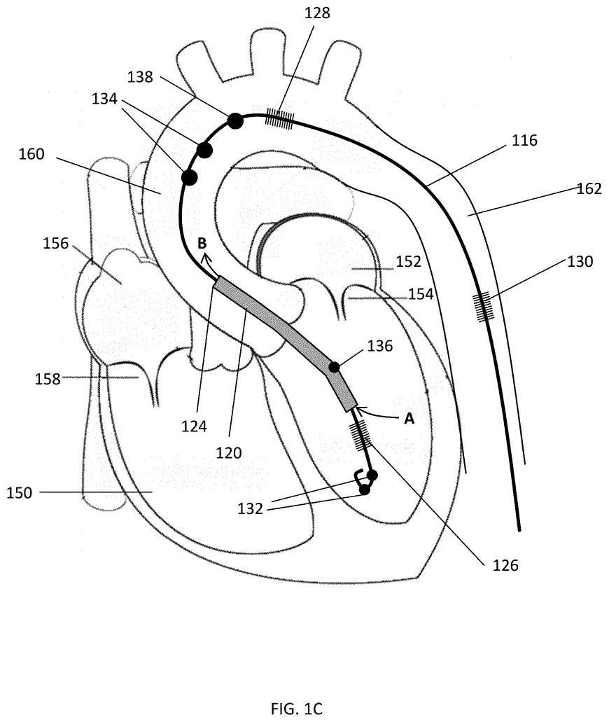

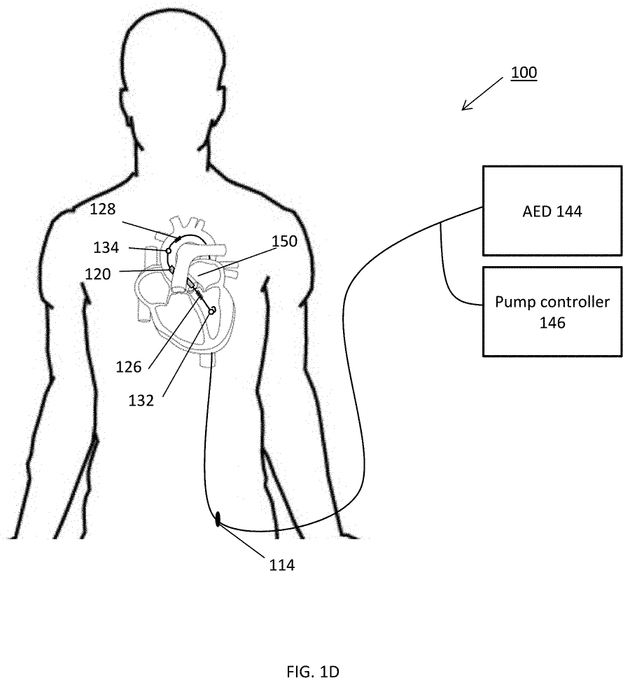

Exemplary embodiments disclosed herein relate to a percutaneous transvenous defibrillating/pacing cardiac assist devices and methods of use. FIG. 1A shows an exemplary illustrative drawing of a defibrillating cardiac assist device (DCAD) 100 and FIGS. 1B-1D show exemplary illustrative drawings of a DCAD 100 implanted in a patient according to some embodiments. As shown in FIG. 1A, a DCAD 100 comprises internal components 110 implanted in the body of a patient, and external components 112 that connect to internal components 110 via a connection port 114. Connection port 114 is shown in FIG. 1B for simplicity as being on the side of the patient, but may exit the body from the femoral artery, as shown in FIG. 1D, or from another convenient point. Internal components 110 include a shared catheter 116, a cardiac assist assembly 102 and a defibrillator assembly 104.

In some embodiments shared catheter 116 is used by both of cardiac assist assembly 102 and defibrillator assembly 104. In some embodiments, cardiac assist assembly 102 includes a flow pump 120, a pump entry port 122 for entry of low-pressure blood from a heart into flow pump 120 and a pump exit port 124 for exit of high-pressure pumped blood from flow pump 120 to the central circulation of the patient. In some embodiments, defibrillator assembly 104 includes an anodal high voltage (HV) defibrillation coil 126 and a first cathodal HV defibrillation coil 128. In some embodiments, defibrillator assembly 104 includes a second cathodal HV defibrillation coil 130. In some embodiments, defibrillator assembly 104 includes a first cardiac rhythm sensor 132 and a second cardiac rhythm sensor 134.

External components 112 comprise a connector cable 140, an automated external defibrillator 144 and a pump controller 146.

As shown in FIGS. 1B and 1C, DCAD 100 is implanted percutaneously and intravenously. The heart 150 of the patient is shown illustratively for simplicity and is not intended to be anatomically correct. Heart 150 comprises left atrium 152, left ventricle 154, right atrium 156 and right ventricle 158. Heart 150 pumps blood into aortic arch 160 and descending aorta 162.

DCAD 100 may be implanted, for example, over a guidewire. As shown in FIG. 1C, pump entry port 122 may be positioned inside left ventricle 152 and pump exit port 124 may be positioned in aortic arch 160, as is pump 120. Alternatively, in some embodiments, DCAD 100 is placed transvenously in the right heart via either the femoral vein, via a transatrial incision, via a transventricular incision or via a route from a great vessel connecting from the superior vena cava such as via the subclavian vein. Alternatively, in some embodiments, DCAD 100 is placed first transvenously and then placed transeptally with the proximal portion of the device in the venous system and the distal portions of the device placed respectively in the left atria, left ventricle and ascending aorta. Therefore, the position of the device as shown in FIG. 1C should not be considered limiting.

In use, pump 120 pumps blood from pump entry port 122 to pump exit port 124 as shown by arrows “A” and “B”. Pump controller 146 is in data communication with pump 120 and controls the operation of pump 120. Data communication between pump 120 and pump controller 146 may use wires (not shown) installed inside catheter 116 and connector cable 140. In some embodiments, flow pump 120 comprises an axial flow motor (not shown).

First cardiac rhythm sensor 132 and second cardiac rhythm sensor 134 of defibrillator assembly 104 are in data communication with an AED 144 which comprises a computing device as defined herein. Anodal HV defibrillation coil 126, first cathodal HV defibrillation coil 128 and second cathodal HV defibrillation coil 130 of defibrillator assembly are in electrical and data communication with AED 144 for providing sensing and defibrillation. Data and electrical communications between the defibrillator assembly 104 and AED 144 may use wires (not shown) integrated within catheter 116 and connector cable 140.

In some embodiments, defibrillator assembly 104 includes a ventricular pressure sensor 136 and a central aortic pressure sensor 138 to enhance sensing fidelity and also to determine if a given arrhythmia is of hemodynamic significance. Pressure sensors 136 and 138 are in data communication with AED 144 via wires (not shown) in catheter 116 and connector cable 140. In some embodiments, ventricular pressure sensor 136 and central aortic pressure sensor 138 comprise electric solid state pressure sensors integrated into catheter 116 such that, when implanted, ventricular pressure sensor 136 is positioned in left ventricle 154 and central aortic pressure sensor 138 is positioned in the aortal arch 160.

In use, when a sustained ventricular arrhythmia is detected based on the signals received by AED 144 from coils 126, 128, and/or 130, and/or sensors 132 and 134 (and also optionally from sensors 136 and 138) AED 144 initiates charging of high voltage capacitors integrated into AED 144. Should the arrhythmia continue for longer than a predetermined period, a high voltage shock will be delivered from AED 144 via the anodal HV coil 126 to either or both of the cathodal HV coils 128 and/or 130 positioned respectively in the aortic arch 160 and descending aorta 162. In some embodiments, a high voltage is between 1500V-1800V. In some embodiments, the delivered shock is a biphasic truncated shock and the vector is a dual vector between both cathodal coils 128 and 130 and anodal coil 126. As shown in FIG. 1A, arrows “C” and “D” show shock and sensing vectors.

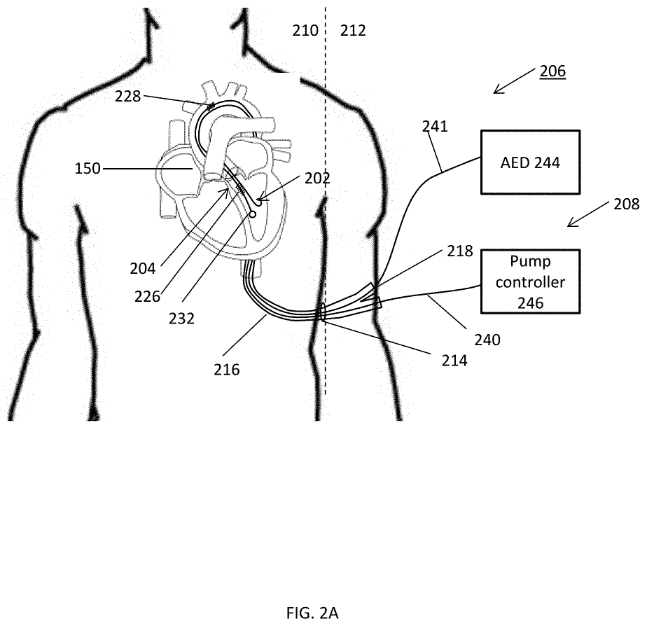

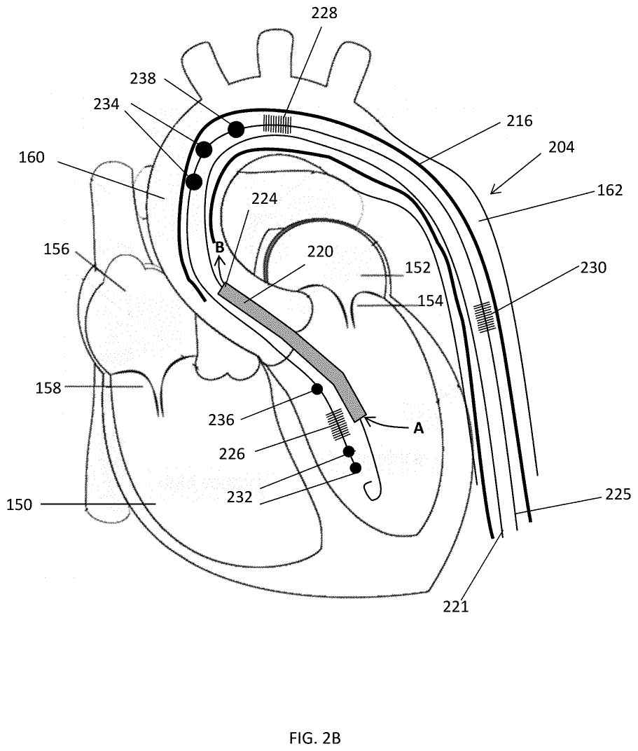

Reference is made to FIGS. 2A and 2B that show exemplary illustrative drawings of an IDP installed into a implanted cardiac assist device according to some embodiments. As shown in FIGS. 2A and 2B, a cardiac assist device 208 is implanted percutaneously and transvenously. Cardiac assist device 208 comprises internal components 210 implanted in the body of a patient and external components 212 that connect to the internal components 210 via a connection port 214. Connection port 214 is shown in FIG. 2A for simplicity as being on the side of a patient but may exit the body from the femoral artery or other convenient point.

In some embodiments, internal components 210 may include a catheter 216 and a cardiac assist assembly 202 that in turn comprises a flow pump 220, a pump entry port 222 for entry of low-pressure blood from a heart into flow pump 220, and a pump exit port 224 for exit of high-pressure pumped blood from flow pump 220 to the central circulation of the patient. External components 212 comprise a connector cable 240, a pump controller 246 and an assist port 218. Assist port 218 enables insertion of other devices into catheter 216.

Cardiac assist assembly 202 may be implanted, for example, over a guidewire such that pump entry port 222 is positioned inside left ventricle 152. Alternative positions are contemplated as described above with reference to FIG. 1C. Pump exit port 224 is positioned in aortic arch 160 as is pump 220. In use, pump 220 pumps blood from pump entry port 222 to pump exit port 224 as shown by arrows “A” and “B”. Pump controller 246 is in data communication with pump 220 and controls the operation of pump 220. Data communication between pump 220 and pump controller 246 may use wires 221 installed inside catheter 216 and connector cable 240.

An IDP 206 using intravascular leads includes a defibrillator assembly 204 a connector cable 241 and an AED 244. Assist port 218 is used for percutaneous and transvenous insertion of a defibrillator assembly 204 to the heart 150 of a patient via catheter 216. In some embodiments, defibrillator assembly 204 includes an anodal HV defibrillation coil 226 and a first cathodal HV defibrillation coil 228. In some embodiments, defibrillator assembly 204 includes a second cathodal HV defibrillation coil 230. In some embodiments, defibrillator assembly 204 includes a first cardiac rhythm sensor 232 and a second cardiac rhythm sensor 234. It should be appreciated that either two shocking coils 226 and 228 or three shocking coils 226, 228 and 230 may be used. In some embodiments, coils 226, 228, 230 are HV coils.

First cardiac rhythm sensor 232 and second cardiac rhythm sensor 234 of defibrillator assembly 204 are in data communication with AED 244 which comprises a computing device as defined herein. Anodal HV defibrillation coil 226, first cathodal HV defibrillation coil 228 and second cathodal HV defibrillation coil 230 of defibrillator assembly 204 are in electrical and data communication with AED 244 for providing sensing and defibrillation. Data and electrical communications between the defibrillator assembly 204 and AED 244 may use wires 225 passed through catheter 216 and running inside connector cable 241.

In some embodiments, defibrillator assembly 204 includes a ventricular pressure sensor 236 and a central aortic pressure sensor 238 to enhance sensing fidelity and also to determine if a given arrhythmia is of hemodynamic significance. Pressure sensors 236 and 238 are in data communication with AED 244 via wires 225 in catheter 216 and connector cable 241. In some embodiments, ventricular pressure sensor 236 and central aortic pressure sensor 238 comprise electric solid-state pressure sensors such that, when implanted, ventricular pressure sensor 236 is positioned in left ventricle 154 and central aortic pressure sensor 238 is positioned in the aortal arch 160.

In use, when a sustained ventricular arrhythmia is detected based on the signals received by AED 244 from coils 226, 228 and/or 230, and/or sensors 232 and 234 (and also optionally from sensors 236 and 238), AED 244 initiates charging of high voltage capacitors integrated into AED 244. Should the arrhythmia continue for longer than a predetermined period, a high voltage shock will be delivered from AED 244 via the anodal HV coil 226 to either or both of the cathodal HV coils 228 and/or 230 positioned respectively in the aortic arch 160 and descending aorta 162. In some embodiments, a high voltage is between 1500V-1800V. In some embodiments, the delivered shock is a biphasic truncated shock and the vector is a dual vector between both cathodal coils 228 and 230 and anodal coil 226.

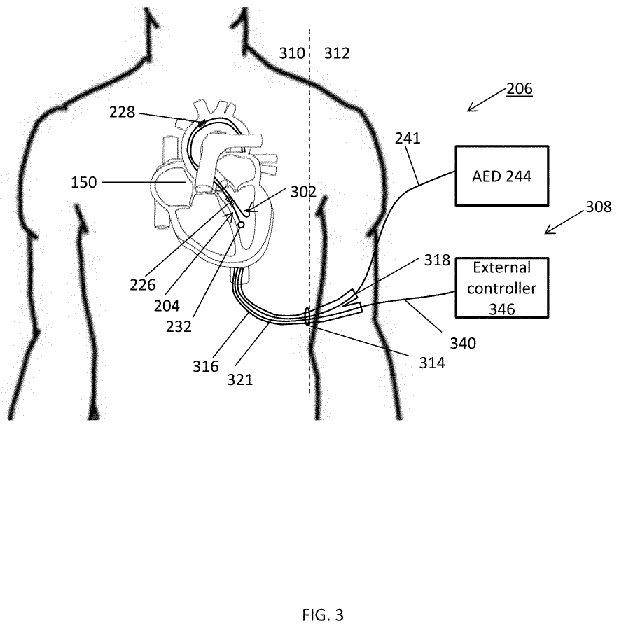

Reference is made to FIG. 3 showing an exemplary illustrative drawing of an IDP installed into an implanted cardiac device. As shown in FIG. 3 an implanted cardiac device 308 is implanted percutaneously and transvenously. Non-limiting examples of cardiac device 308 include a heart monitor, cardiac assist device, cardiac central pressure monitor, cardiac oximetry sensor, cardiac pacing device, and so forth. Cardiac device 308 comprises internal components 310 implanted in the body of a patient and external components 312 that connect to the internal components 310 via a connection port 314. Connection port 314 is shown in FIG. 3 for simplicity as being on the side of a patient but may exit the body from the femoral artery or another convenient point.

Internal components 310 may include a catheter 316 and cardiac assist assembly 302, Catheter 316 enables communication between cardiac assist assembly 302 and external components 312 such as an external controller 346. External components 312 also comprise a connector cable 340 and an assist port 318. Assist port 318 enables insertion of other devices into catheter 316.

Cardiac device 308 is implanted such that a distal end of catheter 316 is positioned in the heart or connecting artery of a patient. Alternative positions are contemplated as described above with reference to FIG. 1C. External controller 346 is in data communication with internal cardiac assist assembly 302 using wires 321 installed inside catheter 316 and connector cable 340.

An IDP 206 using intravascular leads utilizes assist port 318 for percutaneous and transvenous insertion of defibrillator assembly 204 to the heart 150 of a patient via catheter 316. Defibrillator/pacer assembly 204 is described further with reference to FIGS. 2A and 2B above and is in communication with an external AED 244.

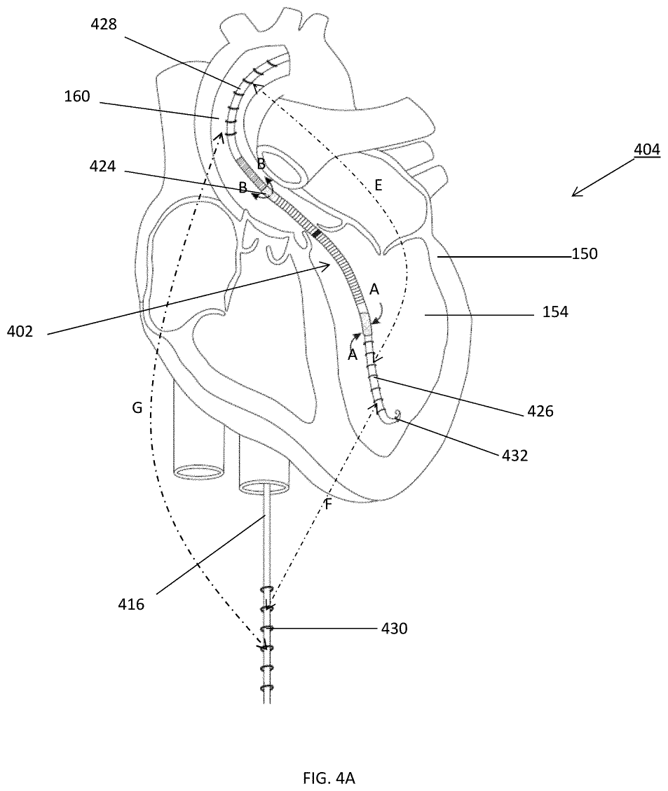

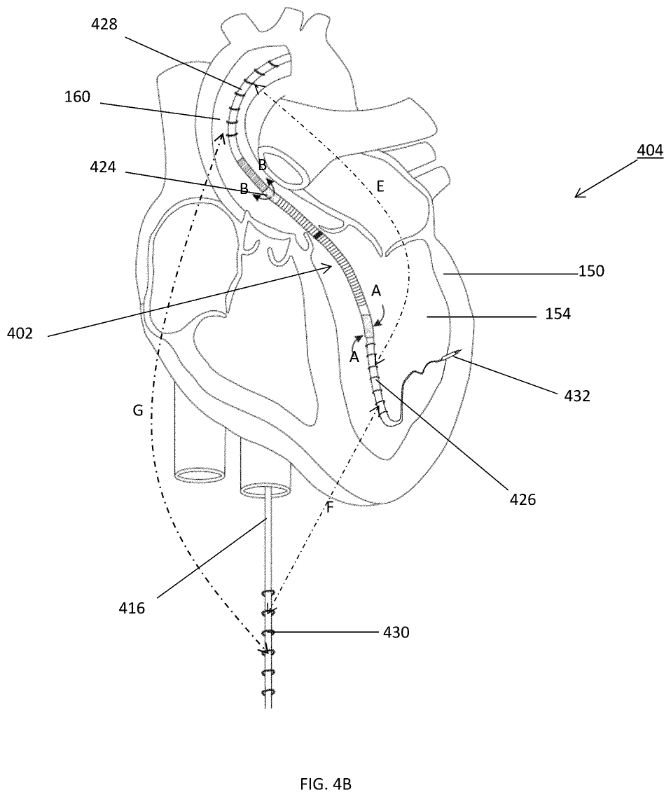

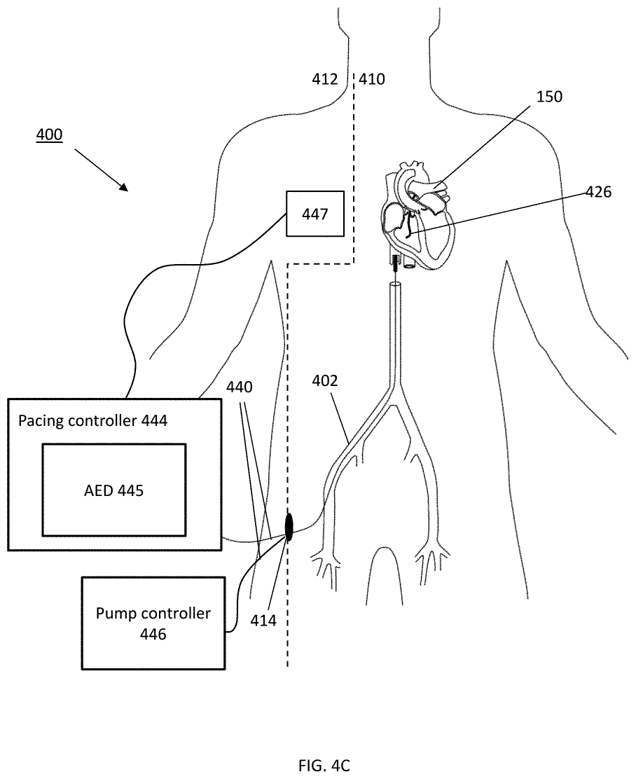

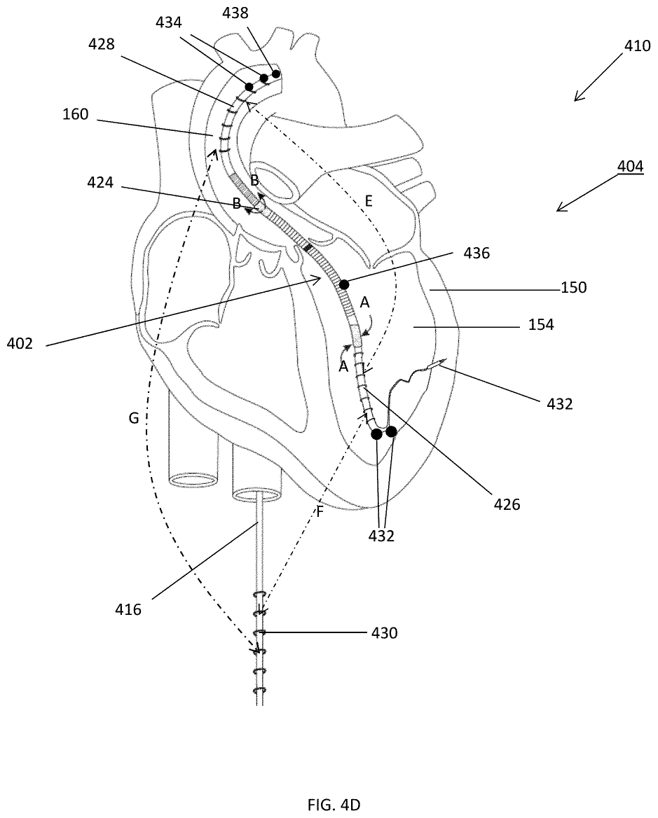

FIGS. 4A-4D show an exemplary illustrative drawings of a pacing cardiac assist device (PCAD) 400 implanted in a patient according to some embodiments. As shown in FIG. 4A, a PCAD 400 comprises internal components 410 implanted in the body of a patient, and external components 412 that connect to internal components 410 via a connection port 414. Connection port 414 is shown in FIG. 4B as connecting into the femoral artery 402 but may be connected at any other part of the body as required.

Internal components 410 may include a shared catheter 416 for use by a cardiac assist assembly 402 and a pacer assembly 404. Non-limiting examples of cardiac assist assembly 402 include a heart monitor, cardiac central pressure monitor, cardiac oximetry sensor and so forth. In some embodiments, such as shown in FIGS. 4A-4B, cardiac assist assembly 402 is a flow pump 420 including a pump entry port 422 for entry of low-pressure blood from a heart into flow pump 420 and a pump exit port 424 for exit of high-pressure pumped blood from flow pump 420 to the central circulation of the patient. In some embodiments, pacer assembly 404 may include a first coil 426 and a second coil 428. In some embodiments, pacer assembly 404 may further include a third coil 430. In some embodiments, coils 426, 428, 430 are HV coils. In some embodiments, coils, 426, 428, 430 and 432 are positioned on PCAD 400 so as to respectively be positioned in the LV of the heart and arteries as shown in FIG. 4A.

In some embodiments, such as shown in FIG. 4B, pacer assembly 404 may further include a pacing lead 432. Pacing lead 432 is positioned so as to be in contact with the cardiac wall. Pacing lead 432 may be unipolar or bipolar. In some embodiments, pacing lead is a screw-in pacing lead. In some embodiments (FIG. 4D), pacer assembly 404 may further include a first cardiac rhythm sensor 432 and a second cardiac rhythm sensor 432.

External components 412 may include a connector cable 440, a pacing controller 444 and a pump controller 446.

As shown in FIGS. 4A-4D, PCAD 400 may be implanted percutaneously and intravenously. The anatomy including the heart 150 of the patient is shown illustratively for simplicity and is not intended to be anatomically correct. PCAD 400 may be implanted, for example, over a guidewire. As shown in FIGS. 4A-4B, pump entry port 422 is positioned inside left ventricle 152 and pump exit port 424 is positioned in aortic arch 160, as is pump 420. Alternatively, in some embodiments (FIG. 4C), a portion of PCAD 400 may be placed transvenously in the right heart via either the femoral vein, via a transatrial incision, via a transventricular incision or via a route from a great vessel connecting from the superior vena cava such as via the subclavian vein. Alternatively, in some embodiments, PCAD 400 may be placed first transvenously and then placed transeptally with the proximal portion of the device in the venous system and the distal portions of the device placed respectively in the left atria, left ventricle and ascending aorta. Therefore, the position of the device as shown in FIGS. 4A-4D should not be considered limiting.

In use, pump 420 pumps blood from pump entry port 422 to pump exit port 424 as shown by arrows “A” and “B”. Pump controller 446 is in data communication with pump 420 and controls the operation of pump 420. Data communication between pump 420 and pump controller 446 may use wires (not shown) installed inside catheter 416 and connector cable 440. In some embodiments, flow pump 420 may comprise an axial flow motor (not shown).

First coil 426, second coil 428, third coil 430, and pacing lead 432 may be referred to herein as “electrodes”. Electrodes 426, 428, 430, and 432 are in electrical and data communication with pacing controller 444. Data and electrical communications between electrodes 426, 428, 430, 432 and pacing controller 444 may use wires (not shown) integrated within catheter 416 and connector cable 440. In some embodiments, pacing controller 444 receives sensing information from electrodes 426, 428, 430, and 432. In some embodiments, pacing controller 444 may generate electrical pulses for pacing via electrodes 426, 428, 430 and 432. In some embodiments, pacing controller 444 and pump controller 446 are combined into a single device. In some embodiments, pacing controller 444 additionally or alternatively receives sensing information from first cardiac rhythm sensor 432, and a second cardiac rhythm sensor 432.

PCAD 400 may be configured to provide several alternative unipolar and bipolar pacing/sensing electrode pairs. In some embodiments, pacing and sensing are provided alternately by the same electrode pair. In some embodiments, pacing and/or sensing may be delivered via a far field bipolar pacing configuration consisting of coils 426 and 428 (shown as vector “E”) or as far field bipoles consisting of coils 426 and 430 (shown as vector “F”). In some embodiments, a combination of different unipolar or bipolar pacing vectors can be used. For example, pacing may occur with: coil 426 as the anode and coil 430 as the cathode; or pacing lead 432 as the anode and coil 428 as the cathode; or pacing lead 432 as the anode and coil 426 as the cathode. In some embodiments, an integrated bipolar configuration may be used including where pacing lead 432 is bipolar and acts as cathode and anode. Advantageously, pacing and/or sensing via these various combinations will also allow for anti-tachycardia overdrive pacing to effectively terminate monomorphic VT without the need of a high voltage shock. In some embodiments, where PCAD 400 is positioned in the left ventricle, pacing may also be used to assist in cardiac resynchronization to treat ventricular desynchrony as found in patients with left bundle branch block. In some embodiments, a bipole including coils 428 and 430 or a bipole including coil 428 and pacing lead 432 may be used for sensing both atrial and ventricular activity. In some embodiments, at least one electrode in a pacing electrode pair must reside in the left ventricle to allow for capture and successful pacing of the heart.

In some embodiments, PCAD 400 includes one or more surface electrodes 447. A surface electrode 447 is attached to the skin of a patient such as with adhesive. Surface electrode 447 is in communication for pacing controller 444. In some embodiments, an electrode pair may include surface electrode 447 and another electrode (426, 428, 430, 432). In some embodiments, an electrode pair may include more than one surface electrode 447. In some embodiments, a common patient ground consisting of a combination of electrodes (428, 430) or a surface electrode 447 could allow for unipolar pacing via electrodes 426 or pacing lead 432.

In some embodiments, a common electrical ground is formed by connecting two or more electrodes (in pacing controller 444) and using this common ground as a reference to another electrode that is not part of the common ground to form an electrode pair.

In some embodiments, pacing controller 444 may enable selection by an operator (such as a medical professional), via an operator interface (not shown), of electrode pairs for pacing and electrode pairs for sensing. In some embodiments, pacing controller 444 may automatically select electrode pairs for pacing and electrode pairs for sensing. In some embodiments, pacing controller 444 may test various electrode pairs automatically and determine the pair with the lowest pacing thresholds and the electrode pair with the best cardiac QRS and/or P wave sensing and thus automatically configure PCAD 400 to optimize one or both of pacing efficiency and sensing reliability.

In some embodiments, ideal pacing efficiency may be determined by pacing controller 444 by evaluating which pacing electrode configuration and anticipative timing results in one or both of maximization of cardiac output or maximization of cardiac synchrony. In some embodiments, cardiac output and/or cardiac synchrony are determined by one or more of echocardiography, cardiac output measurements, venous saturations or other invasive and non-invasive measurements of cardiac hemodynamics and/or cardiac output performed by pacing controller 444 and/or other methods with the results and/or electrode configurations provided to controller 444 via an operator interface (not shown). Pacing efficiency determined by pacing controller 444 may be used to determine ideal electrode pair configurations for pacing/sensing.

In some embodiments, operation of the pacing of PCAD 400 includes building a database in pacing controller 444 (or another external device) of a cardiac cycle of a patient suffering from bundle branch block, and artificially pacing a ventricle of the patient using PCAD 400 according to anticipative atrioventricular (AV) delays in the database which are based on measured P-P intervals in the database. This method of operation is referred to herein as dynamically anticipative resynchronization pacing (DAPR) and is further described in co-invented and co-owned U.S. Pat. No. 9,352,159 titled “Cardiac resynchronization therapy utilizing p-wave sensing and dynamic anticipative left ventricular pacing” which is incorporated herein by reference. DAPR may be delivered via PCAD 400 to allow for A/V synchrony, right bundle branch activation and LV pacing with QRS fusion to optimize cardiac output and synchrony.

Sensing for DAPR may be provided via any of the electrode pairs described above including combinations including surface electrodes 447.

In some embodiments, in a typical DAPR type configuration, coils (for example, coils 426, 428, 430 and 430), may sense the patient's sinus rhythm at various rates such that pacing controller 444 may build a rate table of expected atrio-ventricular conduction times. Once this rate table is built, PCAD 400 may anticipate ventricular conduction after a sensed P wave at a given heart rate. Thus, at a given heart rate, when a P wave is sensed after a given period of delay, based on the derived conduction time and rate table, PCAD 400 may anticipate right bundle branch conduction and, at substantially the same time as anticipated right bundle branch conduction, may deliver LV pacing through any of the various LV unipolar and bipolar pacing electrode pairs (such as described above) available in PCAD 400. It should be appreciated that such an approach may effectively allow for a ventricular fusion complex with right bundle branch conduction and cardiac resynchronization via LV pacing.

Several advantages are contemplated by use of the described PCAD 400 including: 1) maintenance of atrio-ventricular synchrony with every P wave followed by a paced and resynchronized QRS complex; 2) lack of need for a dedicated atrial sensing electrode implanted in the atria, as P wave sensing may occur via the electrode pairs proximal to the atria (such as coil pair 428 and 430 which represent an electrical vector inclusive of bi-atrial activation); and 3) ability to allow for native right bundle branch conduction at the time of LV pacing, thus preserving RV native activation and synchrony (or for a right sided PCAD 400, preservation of left bundle activation and synchrony).

In some embodiments, PCAD 400 may also function as a DPCAD (and thus referred to as DPCAD 400) where pacing controller 444 further includes an AED 445 such as AEDs 144 or 244 described above for providing defibrillation via electrodes 426, 428, 430. Alternatively, AED 445 is a separate device that is also in data and electrical communication with pacing controller 444 and internal components 410. In some embodiments, pacing controller 444 is integrated into AED 445. In some embodiments, DPCAD 400 may thus additionally utilize electrodes 426, 428, and/or 430 to deliver high voltage shock therapy to terminate malignant ventricular and/or atrial arrhythmias such as in the embodiments of FIGS. 1A-1D, 2A-2B, and 3. Pacer assembly 404 may therefore be referred to herein as pacer/defibrillation assembly 404. In some embodiments, DPCAD 400 may combine pacing, sensing, and defibrillation using pacing lead 432 as the anode for pacing/sensing, coil 430 as the cathode for pacing/sensing and coils 426 and 428 for delivery of high voltage shocks for termination of malignant atrial and/or ventricular arrhythmias. As shown in FIGS. 4A and 4B, in some embodiments, vectors E, F and G represent potential bipolar vectors for any of pacing, shocking and sensing.

In some embodiments (FIG. 4D), pacing assembly 404 includes a ventricular pressure sensor 436, and a central aortic pressure sensor 438 to enhance sensing fidelity and also to determine if a given arrhythmia is of hemodynamic significance. Pressure sensors 436 and 438 are in data communication with pacing controller 444 and/or AED 445 via wires (not shown) in catheter 416 and connector cable 440. In some embodiments, ventricular pressure sensor 436, and central aortic pressure sensor 438 comprise electric solid state pressure sensors integrated into catheter 416 such that, when implanted, ventricular pressure sensor 436 is positioned in left ventricle 454 and central aortic pressure sensor 438 is positioned in the aortal arch 160.

In use, when a sustained ventricular arrhythmia is detected based on the sensing received by pacing controller 444 from electrodes 426, 428, 430, 432, and/or sensors 432 and 434 (and also optionally from sensors 436 and 438) AED 445 initiates charging of high voltage capacitors (not shown) integrated into AED 445. Should the arrhythmia continue for longer than a predetermined period, a high voltage shock will be delivered from pacing AED 445 via coil 426 to either or both of the coils 428 and/or 430 (vectors “E” and/or “F”). In some embodiments, the delivered shock is a biphasic truncated shock and the vector is a dual vector between both cathodal coils 428 and 430 and anodal coil 426.

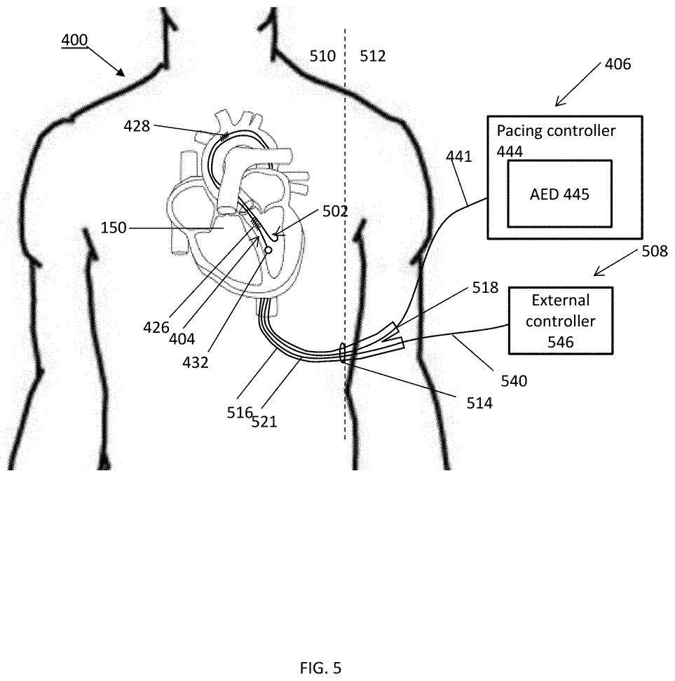

Reference is made to FIG. 5 showing an exemplary illustrative drawing of an IDP installed into an implanted cardiac device. As shown in FIG. 5 an implanted cardiac device 508 is implanted percutaneously and transvenously. Non-limiting examples of cardiac device 508 include a heart monitor, cardiac assist device, cardiac central pressure monitor, cardiac oximetry sensor, cardiac pacing device, and so forth. Cardiac device 508 comprises internal components 510 implanted in the body of a patient and external components 512 that connect to the internal components 510 via a connection port 514. Connection port 514 is shown in FIG. 3 for simplicity as being on the side of a patient but may exit the body from the femoral artery or other convenient point.

Internal components 510 may include a catheter 516 and cardiac assist assembly 502, Catheter 516 enables communication between cardiac assist assembly 502 and external components 512 such as an external controller 546. External components 512 also comprise a connector cable 540 and an assist port 518. Assist port 518 enables insertion of other devices into catheter 516.

Cardiac device 508 is implanted such that a distal end of catheter 516 is positioned in the heart or connecting artery of a patient. Alternative positions are contemplated as described above with reference to FIG. 1C. External controller 546 is in data communication with internal cardiac assist assembly 502 using wires 521 installed inside catheter 516 and connector cable 540.

An IDP 406 using intravascular leads utilizes assist port 518 for percutaneous and transvenous insertion of defibrillator/pacer assembly 404 to the heart 150 of a patient via catheter 516. Defibrillator/pacer assembly 404 is described further with reference to FIGS. 4A-4D above and is in communication with an external AED 445, and pacing controller 444. In some embodiments, AED 445 and pacing controller 444 are an integrated device.

In the claims or specification of the present application, unless otherwise stated, adjectives such as “substantially” and “about” modifying a condition or relationship characteristic of a feature or features of an embodiment, are understood to mean that the condition or characteristic is defined to within tolerances that are acceptable for operation of the embodiment for an application for which it is intended.

Implementation of the method and system of the present disclosure may involve performing or completing certain selected tasks or steps manually, automatically, or a combination thereof. Moreover, according to actual instrumentation and equipment of preferred embodiments of the method and system of the present disclosure, several selected steps may be implemented by hardware (HW) or by software (SW) on any operating system of any firmware, or by a combination thereof. For example, as hardware, selected steps of the disclosure could be implemented as a chip or a circuit. As software or algorithm, selected steps of the disclosure could be implemented as a plurality of software instructions being executed by a computer using any suitable operating system. In any case, selected steps of the method and system of the disclosure could be described as being performed by a data processor, such as a computing platform for executing a plurality of instructions.

Although the present disclosure is described with regard to a “computing device”, a “computer”, or “mobile device”, it should be noted that optionally any device featuring a data processor and the ability to execute one or more instructions may be described as a computer, or computing device including but not limited to any type of personal computer (PC), a server, a distributed server, a virtual server, a cloud computing platform, a cellular telephone, an IP telephone, a smartphone, a smart watch or a PDA (personal digital assistant). Any two or more of such devices in communication with each other may optionally comprise a “network” or a “computer network”.

It should be understood that where the claims or specification refer to “a” or “an” element, such reference is not to be construed as there being only one of those elements.

In the description and claims of the present application, each of the verbs, “comprise” “include” and “have”, and conjugates thereof, are used to indicate that the object or objects of the verb are not necessarily a complete listing of components, elements or parts of the subject or subjects of the verb.

While this disclosure describes a limited number of embodiments, it will be appreciated that many variations, modifications and other applications of such embodiments may be made. The disclosure is to be understood as not limited by the specific embodiments described herein, but only by the scope of the appended claims.

1-6. (canceled)

7. A device, comprising:
a shared catheter;
an automated external defibrillator (AED);
a defibrillator assembly comprising at least one cardiac rhythm sensor in communication with the AED, wherein the at least one cardiac rhythm sensor comprises a first cardiac rhythm sensor and a second cardiac rhythm sensor; and
a cardiac assist assembly, wherein the defibrillator assembly includes at least two defibrillation coils in communication with the AED and wherein the defibrillator assembly and the cardiac assist assembly use the shared catheter for percutaneous and intravenous implantation into a patient.

8-13. (canceled)

14. A device, comprising:
a shared catheter;
a pacing assembly;
a pacing controller; and
a cardiac assist assembly, wherein the pacing assembly includes at least two electrodes in communication with the pacing controller, and wherein the pacer assembly and the cardiac assist assembly use the shared catheter for percutaneous and intravenous implantation into a patient, wherein one of the at least two electrodes is a pacing lead, wherein the device is configured to use the at least two electrodes for both pacing and sensing, and wherein the pacing controller is configured for selecting an electrode pair for pacing and an electrode pair for sensing.

15. (canceled)

16. A device, comprising:
a shared catheter;
a pacing assembly;
a pacing controller;
a cardiac assist assembly, wherein the pacing assembly includes at least two electrodes in communication with the pacing controller, wherein the pacer assembly and the cardiac assist assembly use the shared catheter for percutaneous and intravenous implantation into a patient, and wherein one of the at least two electrodes is a pacing lead; and
an external electrode for adhering to the skin of the patient,
wherein the device is configured to use the at least two electrodes for both pacing and sensing, and
wherein any combination of the at least two electrodes and the external electrode may be used to form a common ground.

17. A device, comprising:
a shared catheter;
a pacing assembly;
a pacing controller;
a cardiac assist assembly, wherein the pacing assembly includes at least two electrodes in communication with the pacing controller, wherein the pacer assembly and the cardiac assist assembly use the shared catheter for percutaneous and intravenous implantation into a patient,
wherein one of the at least two electrodes is a pacing lead,
wherein the device is configured to use the at least two electrodes for both pacing and sensing, and
wherein the device is configured to provide dynamically anticipative resynchronization pacing.

18. A device, comprising:
a shared catheter;
a pacing assembly;
a pacing controller;
a cardiac assist assembly, wherein the pacing assembly includes at least two electrodes in communication with the pacing controller, wherein one of the at least two electrodes is a pacing lead, wherein the pacer assembly and the cardiac assist assembly use the shared catheter for percutaneous and intravenous implantation into a patient, and wherein the device is configured to use the at least two electrodes for both pacing and sensing; and
an automated external defibrillator (AED), wherein at least two of the electrodes are high voltage (HV) electrodes, wherein the AED is in communication with the HV electrodes and configured to use the HV electrodes to provide defibrillation.

19. The device of claim 18, wherein the AED is integrated into the pacing controller or vice versa.

20. The device of claim 19, wherein the pacing assembly further comprises at least one cardiac rhythm sensor in communication with the pacing controller.

21. The device of claim 20, further comprising at least one pressure sensor in communication with the AED.

22. The device of claim 21, wherein the AED is configured to detect a sustained ventricular arrhythmia in the heart of the patient and to deliver a high voltage shock via the HV electrodes when the sustained ventricular arrhythmia is detected.

23. (canceled)

24. The device of claim 20, wherein the at least one cardiac rhythm sensor comprises a first cardiac rhythm sensor and a second cardiac rhythm sensor.

25. A device for use with a cardiac assist device for implantation into a patient, comprising:
a pacing and defibrillation assembly;
an automated external defibrillator (AED); and
a pacing controller,
wherein the AED and pacing controller are in data communication with the pacing and defibrillation assembly for providing sensing, pacing and defibrillation, wherein the cardiac device comprises a catheter and an assist port and wherein the pacing and defibrillation assembly is percutaneously and intravenously implanted into the patient using the catheter and the assist port of the cardiac device.
